SEQUENCE LISTING (20 sequences)

SEQ ID NO: 1 - DNA, 20 bp, Artificial Sequence
  Synthesized
cgttgacatccgtaaagacc

SEQ ID NO: 2 - DNA, 20 bp, Artificial Sequence
  Synthesized
tagagccaccaatccacaca

SEQ ID NO: 3 - DNA, 20 bp, Artificial Sequence
  Synthesized
catgatctaccctccccacg

SEQ ID NO: 4 - DNA, 21 bp, Artificial Sequence
  Synthesized
cagaccaaagacttcctgccc

SEQ ID NO: 5 - DNA, 18 bp, Artificial Sequence
  Synthesized
gcgaactgggccagaaca

SEQ ID NO: 6 - DNA, 19 bp, Artificial Sequence
  Synthesized
ggcctacctgggcaaaatg

SEQ ID NO: 7 - DNA, 24 bp, Artificial Sequence
  Synthesized
cggttccgatgccctgaggctctt

SEQ ID NO: 8 - DNA, 24 bp, Artificial Sequence
  Synthesized
cgtcacacttcatgatggaattga

SEQ ID NO: 9 - DNA, 19 bp, Artificial Sequence
  Synthesized
atgaggctgcggaagaagg

SEQ ID NO: 10 - DNA, 22 bp, Artificial Sequence
  Synthesized
gccgaggaagaggaaaggatag

SEQ ID NO: 11 - DNA, 22 bp, Artificial Sequence
  Synthesized
gtgcaaaagaccctgaacaatg

SEQ ID NO: 12 - DNA, 26 bp, Artificial Sequence
  Synthesized
gaagctattctagtctgataacctcc

SEQ ID NO: 13 - DNA, 18 bp, Artificial Sequence
  Synthesized
gagggctgacgagatagt

SEQ ID NO: 14 - DNA, 18 bp, Artificial Sequence
  Synthesized
actgatgaggctggagat

SEQ ID NO: 15 - DNA, 18 bp, Artificial Sequence
  Synthesized
ggagaggcaaggggattc

SEQ ID NO: 16 - DNA, 20 bp, Artificial Sequence
  Synthesized
ggaggagcaagagaatgtca

SEQ ID NO: 17 - DNA, 18 bp, Artificial Sequence
  Synthesized
gctctagcaagttgttct

SEQ ID NO: 18 - DNA, 18 bp, Artificial Sequence
  Synthesized
aattgcctggcttatctt

SEQ ID NO: 19 - DNA, 18 bp, Artificial Sequence
  Synthesized
tctcttggcaccactttg

SEQ ID NO: 20 - DNA, 18 bp, Artificial Sequence
  Synthesized
ataggggcaggctttact

(19) United States
(12) Patent Application Publication
(10) Pub. No.: US 2019/0070170 A1
(43) Pub. Date: Mar. 7, 2019

(54) METHOD OF SELECTIVELY INHIBITING MPGES-1

(71) Applicant: Macau University of Science and Technology, Taipa (MO)

(72) Inventors: Hua Zhou, Taipa (MO); Jian-Xin Liu, Taipa (MO); Jin-Fang Luo, Taipa (MO); Chun-Song Chen, Taipa (MO); Elaine Lai-Han Leung, Taipa (MO); Liang Liu, Taipa (MO)

(21) Appl. No.: 15/695,304

(22) Filed: Sep. 5, 2017

(51) Int. Cl. A61K 31/485; G01N 33/573; C12Q 1/68

(52) U.S. Cl. CPC A61K 31/485; G01N 33/573; C12Q 1/6883; G01N 2800/50; G01N 2333/99; G01N 2800/32; C12Y 503/99003

(57) ABSTRACT

A method of selectively inhibiting the overexpression of mPGES-1 in a subject in need thereof includes a step of administering an effective amount of a selective mPGES-1 inhibitor or a salt thereof to the subject. A method of treating a subject suffering from a disease associated with an overexpression of mPGES-1 and having a risk of cardiovascular event includes the step of administering an effective amount of a selective mPGES-1 inhibitor to the subject.

### SEQUENCE LISTING

The Sequence Listing file entitled “sequencelisting” having a size of 3,879 bytes and a creation date of Sep. 5, 2017, that was filed with the patent application is incorporated herein by reference in its entirety.

### TECHNICAL FIELD

This invention relates to a method of selectively inhibiting the expression and/or activity of mPGES-1 in a subject. In particular but not exclusively, it relates to a method suitable for treating and/or preventing a subject suffering from a disease associated with an overexpression of mPGES-1.

### BACKGROUND OF THE INVENTION

Chronic inflammation involves a prolonged inflammatory response and is found to have an overexpression of pro-inflammatory proteins and reduced expression of anti-inflammatory proteins. Patients suffering from chronic inflammation are generally required to receive long-term treatment before cure.

Rheumatoid arthritis (RA) is an autoimmune disease characterized by chronic inflammation and damaged joints. Currently, non-steroidal anti-inflammatory drugs (NSAIDs), for example cyclooxygenase (COX)-2 inhibitors, are applied to inhibit the release of prostaglandin (PGE₂). However, reports revealed that the long-term use of these drugs is associated with increased risk of cardiovascular events such as heart attack, stroke and myocardial infarction. It is because the inhibition of COXs activity leads to a destruction in prostaglandin homeostasis, especially prostacyclin (PGI₂) and thromboxane (TX)A₂. Given the adverse effects caused by NSAIDs, some of them were stopped for use in treatment.

Microsomal prostaglandin E synthase 1 (mPGES-1) is a terminal synthase which catalyzes COX-1 and COX-2-derived PGH₂ conversion to PGE₂. Overexpression of mPGES-1 has been found in many chronic immune diseases. However, to date, there is a lack of an effective way to effectively inhibit the overexpression of mPGES-1 for treatment of diseases. There are no agents in the market available for treating diseases via suppressing mPGES-1 expression.

Accordingly, there remains a strong need for developing an effective method for suppressing the overexpression of mPGES-1 in a subject suffering from disease or disorder associated with the overexpression of mPGES-1 and at the same time having a lower risk of cardiovascular event.

### SUMMARY OF THE INVENTION

In a first aspect, the present invention pertains to a method of selectively inhibiting the overexpression of mPGES-1 in a subject in need thereof comprising a step of administering an effective amount of a selective mPGES-1 inhibitor having a structure of Formula (I) or a salt thereof to the subject:

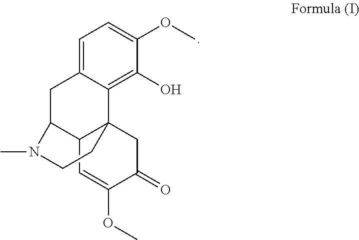

Preferably, the selective mPGES-1 inhibitor is sinomenine having a structure of Formula (II) or a salt thereof:

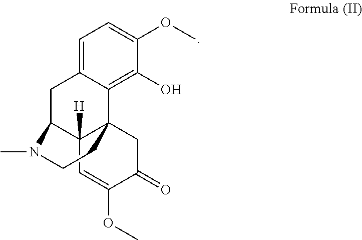

The subject may be suffering from at least one of inflammatory disease, neurological disease, injury, gastrointestinal disease, immune disease, or cancer; or at the same time suffering from a cardiovascular disease.

The subject may be suffering from arthritis and is at a risk of cardiovascular event selected from the group consisting of heart attack, stroke, myocardial infarction, acute coronary syndrome, arteriosclerosis, thrombosis, hypertension, cardiovascular death, and peripheral vascular disease.

In a second aspect of the present invention, there may be provided a pharmaceutical composition comprising a selective mPGES-1 inhibitor having a structure of Formula (I) or a salt thereof:

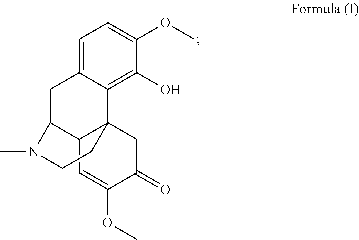

and a non-steroidal anti-inflammatory drug (NSAID) or a salt thereof, wherein the NSAID may be COX-2 inhibitor.

In a third aspect, there is provided a method of treating a subject suffering from a disease associated with an overexpression of mPGES-1 and having a risk of cardiovascular event, comprising the step of administering an effective amount of said pharmaceutical composition to the subject.

In a further aspect, the present invention pertains to a use of the selective mPGES-1 inhibitor having a structure of Formula (I) in the preparation of a medicament for treating and/or preventing disease associated with overexpression of mPGES-1.

Those skilled in the art will appreciate that the invention described herein is susceptible to variations and modifications other than those specifically described. The invention includes all such variations and modifications. The invention also includes all steps and features referred to or indicated in the specification, individually or collectively, and any and all combinations of the steps or features.

Other features and aspects of the invention will become apparent by consideration of the following detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the level of PGE₂ and cell viability of rat peritoneal macrophages (4.5×10⁶ cells for PGE₂ and 1.5×10⁵ cells for cell viability) after pretreatment of sinomenine (SIN) at a concentration of 160, 320 or 640 μM or 0.5 μM DEX for 1 h, followed by stimulation of LPS (1 μg/ml) for another 24 h. Concentrations of PGE₂ in the cell supernatant were analyzed by ELISA kit and cell viability was analyzed with MTT method. *p<0.05 and **p<0.01 between SIN or DEX and LPS alone, n=5 for PGE₂ and n=3 for cell viability.

FIG. 1B shows the level of PGE₂ and cell viability of RAW264.7 cells (4×10⁵ cells for PGE₂ and 1.4×10⁴ cells for cell viability) after pretreatment of SIN at a concentration of 160, 320 or 640 μM or 0.5 μM DEX for 1 h, followed by stimulation of LPS (100 ng/ml) for another 18 h. Concentrations of PGE₂ in the cell supernatant were analyzed by ELISA kit and cell viability was analyzed with MTT method. *p<0.05 and **p<0.01 between SIN or DEX and LPS alone, n=3.

FIG. 1C shows the level of expressions of p-cPLA₂, cPLA₂, COX-1 and COX-2 and mPGES-1 in rat peritoneal macrophages after being treated with SIN or DEX for 1 h and incubated with LPS for 24 h. Total proteins of cells were extracted and analyzed by western blotting. SIN significantly inhibited mPGES-1 protein expression in activated macrophages compared with LPS alone (**p<0.01 between SIN or DEX and LPS alone, n=3 per group), but no influences on p-cPLA₂, cPLA₂, COX-1 and COX-2 expression.

FIG. 1D shows the level of expressions of p-cPLA₂, cPLA₂, COX-1 and COX-2 and mPGES-1 in A549 cells after being treated with IL-1β alone, or with a combination of IL-1β and SIN or DEX for 48 h (*p<0.05, **p<0.01 between SIN or DEX and IL-1β alone, n=3 per group).

FIG. 1E shows the mRNA levels of COX-2 and mPGES-1 in LPS-activated rat peritoneal macrophages obtained from qRT-PCR analysis. Results revealed that SIN is capable of selectively suppressing mPGES-1 expression (*p<0.05 and **p<0.01 between SIN or DEX and LPS alone, n=4 for COX-2 and n=3 for mPGES-1).

FIG. 1F shows the relative mRNA levels of mPGES-1 and levels of PGE₂ detected by qRT-PCR after an RNA interference experiment. The levels of PGE₂ in culture medium were analyzed by ELISA kit (*p<0.05, **p<0.01 between normal or SIN or DEX and LPS alone (NS siRNA), n=3 per group; # p<0.05 between normal or SIN or DEX and LPS alone (mPGES-1 siRNA), n=3 per group).

FIG. 1G shows the levels of PGI₂, TXA₂ and PGD₂ in culture supernatants of LPS-activated rat peritoneal macrophages. SIN does not show observable effects on the production of PGI₂, TXA₂ and PGD₂ (*p<0.05 and **p<0.01 between SIN or DEX and LPS alone, n=5 per group).

FIG. 2A shows the percentage of increase in paw volume in carrageenan-induced rat paw edema model. SIN pretreatment (i.p) obviously alleviated the swelling of the right hind paw after injection with λ-carrageenan (0.1%, w/v) in a dose dependent manner at 2, 3 and 4 h compared to the vehicle-treated rats. Positive drug DEX (i.p.) also significantly inhibited λ-carrageenan-induced rat paw edema. Data are presented as mean±SEM (n=18 per group) and analysis used a one-way ANOVA with a LED post hoc test, ^{##}p<0.01 between vehicle group and normal group, *p<0.05 and **p<0.01 between three doses SIN groups and vehicle group, and between DEX group and vehicle group.

FIG. 2B shows the levels of COX-1, COX-2 and mPGES-1 in microsomes isolated from the inflamed paw of carrageenan-induced rats. The protein levels of mPGES-1 in the right hind paw of vehicle-treated rats obviously increased compared with normal rats (^{#}p<0.05, n=6 per group). SIN (100 mg/kg) pretreatment significantly down-regulated mPGES-1 expression in right hind paws compared to the vehicle-treated rats (*p<0.05, n=6). Positive drug DEX also remarkably decreased mPGES-1 protein levels in inflamed paw compared with vehicle rats (*p<0.05, n=6). SIN and DEX pretreatments did not produce observable effects on COX-1 and COX-2 levels. Horizontal bars represent median values and analysis using a one-way ANOVA with a LSD post hoc test.

FIG. 2C shows the incidence of arthritis, the average thickness of the hind paws and the total arthritis score of four paws in collagen-induced arthritis DBA/1 mice. Results for the thickness and arthritic score are shown as mean±SEM (n=9 for control, n=10 for model and SIN-treated group, n=11 for MTX-treated group). Analysis used a one-way ANOVA with a LED post hoc test, ^{#}p<0.05, ^{##}p<0.01 between model and controlled groups, *p<0.05 and **p<0.01 between SIN-treated and model groups, and between MTX-treated and model groups.

FIG. 2D shows the levels of mPGES-1, COX-1 and COX-2 protein expressions in hind paws of collagen-induced arthritis DBA/1 mice. The results showed that SIN decreased mPGES-1 protein expression compared to the controlled ones (*p<0.05, n=6 per group). SIN did not produce observable changes in COX-1 and COX-2 expressions in the paw tissues. Data are displayed at mean±SEM (n=6 per group). Analysis used a one-way ANOVA with a LED post hoc test, ^{#}p<0.05 between model group and control group, *p<0.05 between SIN-treated and model group of animals.

FIG. 3A to 3H shows the levels of p-JNK (Thr183/Tyr185), JNK, p-p38 (Thr180/Tyr182), p38, p-ERK (Thr202/Tyr204), ERK, p-c-Jun (Ser63/Ser73), c-Jun, p-CREB (Ser133), CREB protein expressions in rat peritoneal macrophages pretreated with SIN for 1 h followed by incubation with LPS (1 μg/ml) for another 15 min (FIG. 3A to 3F) or 1 h (FIGS. 3G and 3H). *p<0.05 and **p<0.01 between SIN and LPS alone, n=4 (FIG. 3A-3C), n=3 (FIG. 3D-3H). All data are as shown mean±SEM and analyzed using a one-way ANOVA with a LED post hoc test.

FIG. 4A show the levels of p-C/EBPβ (Ser105/T235+T188) protein expression in rat peritoneal macrophages treated with SIN for 1 h before incubation with LPS for 30 min.

FIG. 4B shows the levels of cytosolic C/EBPβ and nuclear C/EBPβ in rat peritoneal macrophages treated with SIN for 1 h before incubation with LPS for 2 h.

FIG. 4C shows the immunofluorescence images of rat peritoneal macrophages after treatment of SIN followed by LPS stimulation for 2 h. C/EBPβ was shown in red in nucleus. Results showed that SIN pretreatment down-regulated CEBPβ protein expression in nucleus.

FIG. 4D shows the results obtained from ChIP assay on the regulation of CEBPβ on mPGES-1 and COX-2 promoters in LPS-stimulated rat peritoneal macrophages. The DNA binding of CEBPβ both in mPGES-1 and COX-2 promoters were reduced after treatment of SIN. Data are expressed at mean±SD (n=3 per group). Analysis used an Independent-Samples T test, *p<0.05.

FIG. 5A to 5C show the levels of p-IKKα, p-IκBα and p-p65, markers for NF-κB signaling pathway, in rat peritoneal macrophages after treatment of SIN for 1 h before LPS incubation for 15 min. Total proteins of cells were extracted and analyzed by western blotting. (*p<0.05 between SIN and LPS alone, n=3 per group). Data are as shown mean and SEM and analyzed using a one-way ANOVA with a LED post hoc test.

FIGS. 5D and 5E show the immunofluorescence images and plot for detection of p65 in rat peritoneal macrophages or RAW264.7 cells respectively after treatment of SIN. P65 was shown in red. In inactivated macrophages (control group), p65 was found surrounding the nuclei which were dyed in blue. After stimulation with LPS for 30 min (LPS group), most p65 was translocated into the nuclei (totally overlay of the red and blue). SIN did not produce observable effects on the nuclear translation of p65 in both LPS-stimulated rat peritoneal macrophages and RAW264.7 cells. The amounts of cells with p65 in the nucleus were counted in each picture of these different groups and the percent was obtained respectively. Results are showed as mean±SEM (n=15-18 pictures obtained from three dependent experiments, per group), analysis used a one-way ANOVA with LSD's post hoc test, **p<0.01.

FIG. 5F shows the levels of cytosolic p65 and nuclear p65 in rat peritoneal macrophages with or without LPS stimulation for 30 min. The results showed that SIN did not suppress the LPS-induced p65 protein expression in the nucleus.

FIG. 5G shows the inhibitory effects of SIN on LPS-induced NF-κB DNA binding activity in rat peritoneal macrophages via EMSA analysis.

FIG. 5H shows the SIN has inhibitory effects on DNA binding number of NF-κB in mPGES-1 promoter but not on COX-2 promoter in LPS-stimulated rat peritoneal macrophages via ChIP assay. Data are expressed at mean±SEM (n=3 per group). Analysis used an Independent-Samples T test, *p<0.05.

FIG. 6A shows the levels of pro-inflammatory mediator NO and iNOS/β in rat peritoneal macrophages after pretreatment with SIN for 1 h and LPS stimulation (1 μg/ml) for 24 h. The production of NO in culture medium was detected using Griess reagent. The total cell lysates were obtained and iNOS protein levels were tested by immunoblotting. (*p<0.05 and **p<0.01 between SIN or DEX and LPS alone, n=4 for Nitrite, n=3 for iNOS, respectively). All data are as shown mean±SEM and analyzed using a one-way ANOVA with a LED post hoc test.

FIG. 6B shows the levels of pro-inflammatory mediator NO and iNOS/β in RAW264.7 cells after pretreatment with SIN for 1 h and LPS incubation (100 ng/ml) for 18 h. The levels of NO in culture medium were detected using Griess reagent. The total cell lysates were obtained and iNOS protein levels were tested by immunoblotting. (*p<0.05 and **p<0.01 between SIN or DEX and LPS alone, n=4 for Nitrite, n=3 for iNOS, respectively). All data are as shown mean±SEM and analyzed using a one-way ANOVA with a LED post hoc test.

FIG. 6C shows the levels of TNF-α and IL-6 in rat peritoneal macrophages after pretreatment with SIN for 1 h and LPS stimulation (1 μg/ml) for 24 h. The levels of TNF-α and IL-6 were detected using ELISA kits. **p<0.01 between SIN or DEX and LPS alone, n=5 per group.

FIG. 6D shows the levels of TNF-α in RAW264.7 cells after pretreatment with SIN for 1 h and LPS incubation (100 ng/ml) for 18 h. The levels of TNF-α were detected using ELISA kit. **p<0.01 between SIN or DEX and LPS alone, n=5 per group. All data are as shown as mean±SEM and analyzed using a one-way ANOVA with a LED post hoc test.

### DETAILED DESCRIPTION OF THE EMBODIMENTS

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usually and unavoidable impurities such as side products and components usually resulting from the respective preparation or method for obtaining the material such as traces of further components or solvents. “Consisting of” means that the material solely consists of, i.e. is formed by the respective element. As used herein, the forms “a,” “an,” and “the,” are intended to include the singular and plural forms unless the context clearly indicates otherwise.

The present invention in the first aspect provides a method of selectively inhibiting the overexpression of mPGES-1 in a subject in need thereof. The method comprises a step of administering an effective amount of a selective mPGES-1 inhibitor or a salt thereof to the subject, preferably the selective mPGES-1 inhibitor has a structure of Formula (I)

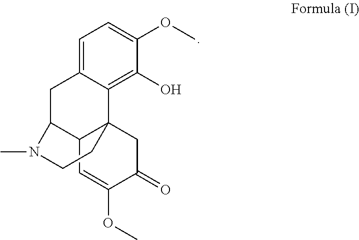

Microsomal prostaglandin E synthase-1 (mPGES-1) is an enzyme which is capable of catalyzing the conversion from cyclooxygenase in particular COX-1 and COX-2 derived prostaglandin H₂ (PGH₂) to prostaglandin (PGE₂), i.e. it can induce the production of PGE₂. The present invention provides a method to selectively target mPGES-1 and/or to inhibit the overexpression of mPGES-1 in a subject. Such an inhibition may be useful to treat and/or prevent diseases or symptoms associated with the overexpression of mPGES-1 in the subject. In particular, a selective mPGES-1 inhibitor of Formula (I) is used in the method.

The term “selective mPGES-1 inhibitor” as used herein refers to a substance which is capable of inhibiting or suppressing the enhanced expression and/or functional activity of mPGES-1 in cells or subject but does not have any or significant inhibitory effect on the expression of cyclooxygenase in particular COX-2. Without intending to be limited by theory, in an embodiment, the selective mPGES-1 inhibitor of the present invention may selectively suppress the binding of nuclear factor KB (NF-κB) to mPGES-1 promoter. The inventors believes that since the selective mPGES-1 inhibitor does not have significant or have no effect on the expression of cyclooxygenase, the prostaglandin homeostasis in cells or subject can be maintained in a relatively stable manner, thereby alleviating diseases or symptoms associated with the overexpression of mPGES-1 and at the same time with a reduced risk of suffering from cardiovascular event.

As used herein, the term “cardiovascular event” means event that is harmful to the heart or blood vessel of a subject. The cardiovascular event may be fatal or non-fatal, and may be, but not limited to, heart attack, stroke, myocardial infarction, acute coronary syndrome, arteriosclerosis, thrombosis, hypertension, cardiovascular death, peripheral vascular disease or the like. In particular, a subject who receives or received long-term treatment of non-steroidal anti-inflammatory drugs (NSAIDs) especially COX-2 inhibitor may be at risk, or high risk, of cardiovascular event. Long-term treatment may last from 3 months to more than 1 year, or may last for an indeterminate length. A patient who is suffering from cardiovascular disease is considered being at a high risk of adverse cardiovascular event if the patient is at the same time administered with COX-2 inhibitor. COX-2 inhibitor is a type of NSAID that directly targets and inhibits the activity of COX-2. The currently available COX-2 inhibitors are found to have significant adverse cardiac side effects.

In an embodiment, the selective mPGES-1 inhibitor of the present invention is administered to a subject at a risk of cardiovascular event, or at a high risk of cardiovascular event. In another embodiment, the selective mPGES-1 inhibitor is administered to a subject suffering from a cardiovascular disease, i.e. the subject has an overexpression of mPGES-1 and suffers from a cardiovascular disease. “High risk” means a risk higher than the average risk of the population at a given age. The person skilled in the art is able to assess the risk of cardiovascular event according to existing clinical guidelines taking age, gender, race, life habit, level of total cholesterol, level of high-density lipoprotein (HDL) cholesterol, systolic blood pressure, occurrence of disease, or the like of a subject into account. Published risk scores may be used as reference.

“Overexpression”, “enhanced expression”, or “enhanced functional activity” preferably means an increase in mPGES-1 expression by at least 5% compared to a reference control, i.e. normal (healthy) cells or subject. The skilled person is able to determine the level of the expression of mPGES-1 in cells or subject. For instance, well-known immunological assays using antibody, in-situ hybridization, qRT-PCR, or similar techniques may be applied to determine the level of the expression of mPGES-1. The inhibition of the overexpression or enhanced functional activity of mPGES-1 may be determined by using Western blotting analysis or other known immunological assays. Preferably, the inhibition is determined by comparing with the level of expression or functional activity of mPGES-1 in a subject before administering the selective mPGES-1 inhibitor.

The subject can be an animal or a human, in particular a mammal. Most preferably, the subject is a human. In an embodiment, the subject is a mammal having an overexpression of mPGES-1, and the overexpression is associated with an inflammatory disease, neurological disease, injury, immune disease, gastrointestinal disease, cancer, or the like. In particular, the subject is suffering from at least one of inflammatory disease, neurological disease, injury, immune disease, gastrointestinal disease, or cancer. The subject may be suffering from at least one of inflammatory disease, neurological disease, injury, gastrointestinal disease, immune disease, or cancer, and a cardiovascular disease or disorder. In an embodiment, the subject may be suffering from at least one of neurological disease, injury, gastrointestinal disease or cancer, and a cardiovascular disease or disorder. In a further embodiment, the subject is suffering from an inflammatory disease in particular arthritis and is at a risk or high risk of cardiovascular event as described above. The subject may be suffering from rheumatic arthritis with an overexpression of mPGES-1 and at the same time a cardiovascular disorder such as high blood pressure, atherosclerosis, thrombosis or the like.

Turning back to the selective mPGES-1 inhibitor, it preferably has a structure of Formula (I), or a salt thereof:

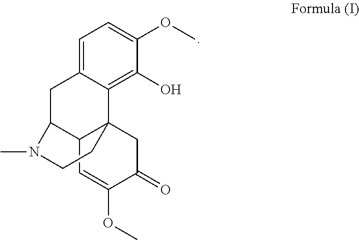

In an embodiment, the selective mPGES-1 inhibitor is sinomenine (also denoted as SIN) and has a structure of Formula (II), or a salt thereof:

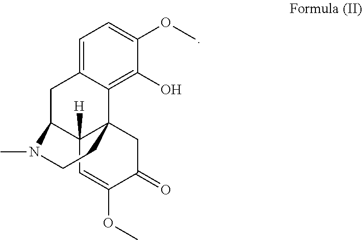

The “salt” refers to an acceptable salt for administration to a subject, i.e. a pharmaceutically acceptable salt. The salt may be prepared based on administration route or dosage regime. Embodiments of salt include the corresponding acid addition salts and organic salts, in particular, but not limiting to, a salt formed by reaction with hydrochloric acid, hydrobromic acid, sulphuric acid, acetic acid, citric acid, oxalic acid, phosphoric acid, succinic acid, carboxylic acid, sulfonic acid, lactic acid or the like.

It is also appreciated that derivatives of the selective mPGES-1 inhibitor as described herein may also be applicable in the method of the present invention.

The expression “effective amount” as used herein generally denotes an amount sufficient to produce therapeutically desirable results, i.e. it means a therapeutically effective amount. The exact nature of the result may vary depending on the specific disease or disorder which is targeted. When the disease is an inflammatory disease, the result may be an inhibition or reduction of pro-inflammatory protein markers, an increase of anti-inflammatory markers or the amelioration of symptoms related to the inflammation. According to the present invention, it is preferably an amelioration of symptoms associated with mPGES-1 or reduction in the level of expression of PGE₂, thereby alleviating inflammatory symptoms in particular those caused by the increase of PGE₂ level.

The method of the present invention may further include steps carried out before administering the selective mPGES-1 inhibitor to the subject, comprising:
obtaining a sample such as plasma or synovial lining cells from the subject;
testing said sample for the expression of mPGES-1;
comparing the level of mPGES-1 expression with a reference to determine if the subject has an overexpression of mPGES-1; and
optionally determining if the subject is suffering from a cardiovascular disease or being at risk of a cardiovascular event.

In another aspect of the present invention, there may be provided a pharmaceutical composition comprising a selective mPGES-1 inhibitor as described above, and at least one of a NSAID in particular a COX-2 inhibitor for treating and/or preventing a disease associated with an overexpression of mPGES-1. In particular, the disease may be inflammatory disease, neurological disease, injury, immune disease, gastrointestinal disease, cancer, or the like. In an embodiment, the disease may be arthritis in particular rheumatoid arthritis. Without intending to be limited by theory, the selective mPGES-1 inhibitor of the present invention may achieve the suppression of PGE₂ via a different mechanism or pathway compared to COX-2 inhibitor. Accordingly, the application of the selective mPGES-1 inhibitor in a combination with other possible COX-2 inhibitor may help to reduce the risk of cardiovascular event triggered by the full dose of COX-2 inhibitor alone. It may be a possible approach to reduce the risk of cardiovascular event while treating a subject suffering from inflammatory disease such as arthritis.

In an embodiment, the pharmaceutical composition may further comprise a pharmaceutically acceptable excipient such as a carrier or diluent that does not have therapeutic activity in a subject. The person skilled in the art is able to select suitable pharmaceutically acceptable excipient when preparing the pharmaceutical composition based on the dosage regime and administration route.

The pharmaceutical composition may comprise:
the selective mPGES-1 inhibitor having the structure of Formula (I), or a salt thereof:

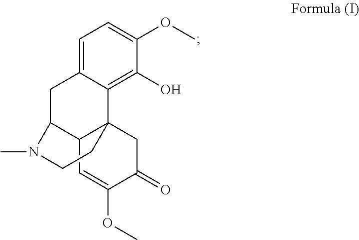

and
a COX-2 inhibitor or a salt thereof, wherein the COX-2 inhibitor may be selected from rofecoxib, ibuprofen, indomethacin, diclofenac, oxaprozin, piroxicam, celecoxibm, or the like.

The selective mPGES-1 inhibitor is as described above. In an embodiment, the selective mPGES-1 inhibitor is sinomenine having a structure of Formula (II) or a salt thereof

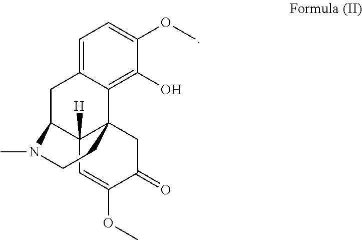

The present invention also pertains to a use of the selective mPGES-1 inhibitor having a structure of Formula (I) in the preparation of a medicament for treating and/or preventing disease associated with overexpression of mPGES-1. The disease associated with overexpression of mPGES-1 is as described above. Further, the medicament prepared possesses a reduced risk of cardiovascular event compared to the one containing the sole active ingredient of NSAID, in particular a COX-2 inhibitor as described above.

In a further aspect, the present invention also relates to a use of the selective mPGES-1 inhibitor having a structure of Formula (I) in the treatment and/or prevention of disease associated with overexpression of mPGES-1, where the disease is selected from neurological disease, injury, gastrointestinal disease, or cancer.

Accordingly, the present invention provides an improved approach for selectively inhibiting the overexpression of mPGES-1 without affecting the expression of cyclooxygenase, thereby alleviating symptoms in particular those associated with inflammation and without significant risk to cardiovascular event. The method and pharmaceutical composition as described herein may exert promising therapeutic effect in treatment or prevention of disease or symptoms highly associated with mPGES-1. Further, the present invention is suitable for patients who are suffering from a cardiovascular disease. It would be also appreciated that the method and pharmaceutical composition as disclosed herein are also useful in research and clinical studies.

### EXAMPLES

### Materials and Methods

1. Chemical Reagents and Antibodies

Lipopolysaccharide (LPS, Escherichia coli 055: B5) and dexamethason (DEX) were purchased from Sigma Chemical Co. (St. Louis, Mo., USA). Sinomenine (SIN) (purity>99%) for cell experiments was obtained from Chengdu Si Ke Hua biological technology Co. LTD (Cheng du, Sichuan Province, China), which was dissolved in DMSO. SIN (purity>99%) for animal experiments was kindly provided by Hunan Zhengqing Pharmaceutical Group Limited (Huaihua, Hunan Province, China).

Antibodies against COX-1, COX-2, cPLA₂, p-cPLA₂, p-IKK-α/β (Ser176/180), IKK-β, p-IκBα (Ser32/36), IκBα, p-p65 (Ser536), p65, iNOS, p-JNK (Thr183/Tyr185), JNK, p-p38 (Thr180/Tyr182), p38, p-ERK (Thr202/Tyr204), ERK, p-CREB (Ser133) and p-CREB were obtained from Cell Signaling Technology (Boston, Mass., USA). Antibodies against p-C/EBPβ (Ser105), C/EBPβ, GAPDH, β-actin and p65 (for ChIP assay) were obtained from Santa Cruz Biotechnology (Santa Cruz, Calif., USA). The monoclonal antibody against mPGES-1 and prostaglandin E₂ (PGE₂), 6-keto Prostaglandin F1α (PGI₂), 11-dehydro Thromboxane B₂ (TXA₂) and prostaglandin D₂ (PGD₂) EIA Kits were obtained from Cayman Chemical (Ann Arbor, Mich., USA). Antibodies against p-CEBPβ (phospho T235+T188), TBP and Lamin B1 were from Abcam (Cambridge, UK). The IRDye 800CW goat anti-mouse IgG (H+L) and IRDye 800CW goat anti-rabbit IgG (H+L) secondary antibodies were purchased from Li-COR Biotechnology (Lincoln, Nebr., USA). ON-TARGETplus SMARTpool mPGES-1 siRNA or nonspecific siRNA were obtained from GE Dharmacon (Lafayette, Colo., USA)/Thermo Scientific (Waltham, Mass., USA), HiPerFect transfection reagent were purchased from QIAGEN (Hilden, Germany).

2. Cell Lines and Cell Cultures

RAW264.7 and A549 cell lines were obtained from American Type Culture Collection (ATCC, Manassas, Va., USA) and maintained in DMEM (RAW264.7) or 1640 (A549) medium containing 10% FBS (Gibco-BRL, Grand Island, N.Y., USA) and antibiotics at 37° C. in a humidified atmosphere containing 5% CO₂. RAW264.7 cells were plated in 6-well plates at a density of 4×10⁵ cells and incubated for 24 h, then the cells were pretreated with different concentrations of SIN for 1 h before stimulating with LPS (100 ng/ml). A549 cells were seeded in 6-well plates at a density of 1×10⁵ cells and incubated for 24 h, followed by pretreatment with SIN. A549 cells were then stimulated by IL-1β (1 ng/ml).

Sprague-Dawley (SD) rats from the University of Hong Kong were housed in cages with free access to food and water. Peritoneal macrophages of SD rats were isolated as described in Liu J. et al., Pharmacological research 2016, 111:303-315. Briefly, untreated rats were sacrificed and after laparotomy, about 50 ml cold sterile Hank's balanced salt solution (HBSS) was used to lavage the peritoneal cavity at twice, and then the peritoneal lavage fluid was collected and centrifuged at 1500 rpm for 10 min. The cell pellets was suspended at a density 1.5×10⁶ cells/ml in pre-hearted DMEM medium with 10% heat-inactivated FBS, penicillin G (100 units/ml), streptomycin (100 mg/ml), and L-glutamine (2 mM), followed by seeding in 6-well plates at a density of 4.5×10⁶ cells and incubation for 2 h. Then the medium were discarded to remove the non-adherent cells. The adherent cells were further washed for two times with pre-heated DMEM medium to remove floating cells. Next, cells were pre-treated with various concentration of SIN for 1 h, followed by stimulation with LPS (1 μg/ml).

3. Animal Models

Rats Having Carrageenan-Induced Paw Edema

The model of carrageenan-induced paw edema was performed with SD rats (150-200 g). The carrageenan-induced paw edema was performed in SD rats (150-200 g) by subcutaneously injection λ-carrageenan as described in Liu J. et al., Pharmacological research 2016, 111 and Luo P. et al., J. Pharmacol. Exp. Ther. 2010. Briefly, rats were fasted for 12 h before experiment and intraperitoneal injection was performed with three different doses (25, 50 and 100 mg/kg) of SIN, or DEX (reference drug, 2 mg/kg) or 0.9% saline (Vehicle and Normal group rats), at 2 h prior to the induction of paw edema. Paw edema was induced by subcutaneous injection of 100 μl of 1% (w/v) freshly prepared λ-carrageenan (Sigma, St Louis, Mo., USA) diluted in saline in the right hind foot pad. 100 μl sterile saline was injected to the rats in the right hind paw as normal control. Paw volumes (ml) of right hind foot of each rat were measured using a plethysmometer (type 7150; UGO Basile, Comerio, Italy) at 0 h (before carrageenan injection) and then again at 1, 2, 3 and 4 h after the injection of carrageenan or saline. The percentage of paw edema of the right hind paws of all groups were calculated at different time points by the following equation:

Swelling ratio in each time points=(the paw volume after injection−the paw volume before injection)/the paw volume before injection×100

After accomplish the experiment, rats were sacrificed by injection of dorminal, which contains 20% pentobarbital and followed by cervical dislocation. The paw tissues were removed and freshly frozen in liquid nitrogen immediately and stored at −80° C.

CIA Model in DBA Mice

Female DBA/1 mice (8-9 weeks old) were purchased from Shanghai SiLaike (SLAC) Laboratory Animal Company (Shanghai, China) and fed with a chow diet and water at room temperature. Equal volume of complete Freuend's adjuvant (CFA) and Bovine Type II Collagen (CII) were mixed and emulsified using a homogenizer (13,000 rpm) on ice. On day 0, DBA/1 mice were immunized by injecting 50 μl of CII in CFA in the tail, approximately 2 cm from the base of the tail. After injection, on day 18, mice with one or more than one paw inflamed were chosen and randomly divided into model group, SIN (100 mg/kg, i.p.) treatment group and MTX (10 mg/kg/week, p.o) treatment group, the mice without immunization were used as normal control. On day 21, these chosen mice were boosted through injecting the same volume of CII in IFA same as the day 0 immunization procedure. The incidence of joint swelling in each group was recorded every two days since drug treatment. Paw thickness (at the ankle joints of the hind paws) of each mouse was obtained by using vernier caliper to record the severity of paw swelling.

Arthritis score of each paw was evaluated every two days during experiment with the following criteria:
0: no evidence of erythema and swelling;
1: erythema and mild swelling confirmed to the tarsals or ankle joint;
2: erythema and mild swelling extending from the ankle to the tarsal joints;
3: erythema and moderate swelling extending from the ankle to metatarsal joints; and
4: erythema and severe swelling encompass the ankle, foot and digits, or ankylosis of the limb.

The score of each paw was summed for a score of 0-16 for each mouse. Mice were then sacrificed and hind paws were removed and freshly frozen in liquid nitrogen and stored at −80° C. until used.

4. Extraction of Microsomes

Frozen paws were pulverized in liquid nitrogen using a stainless steel mortar and pestle, then re-suspended in 8 volumes of ice-cold PBS from Invitrogen (San Diego, Calif., USA) with 2.6 mM DTT from Promega (Madison, Wis., USA) and 2×Complete Protease Inhibitor mixture from Roche (Mannheim, Germany), and homogenized on ice bath using a tissue homogenizer from T25 digital, IKA (Stanfen, Germany) at 10,000 rpm/min for 5 min. Rat paws homogenates were subjected to centrifuged at 10,000×g at 4° C. for 10 min, and the supernatant was filtered using 0.2 μm syringe filter (Pall Corporation), subsequently filtrate was transferred to an ultra-high speed centrifuge tube from Thermo Scientific (Asheville, N.C., USA) and further centrifuged at 50,000×rpm at 4° C. for 90 min. The cell pellets (microsomes) were dissolved in cold PBS (containing 2.6 mM DTT and 2× Complete Protease Inhibitor mixture). The protein concentrations were measured using a Bio-Rad protein assay kit (Hercules, Calif., USA). The sample of microsomal fraction was kept at −80° C. until western blot analysis.

5. Protein Preparation and Western Blotting Analysis

Whole-cell and nuclear proteins were obtained using RIPA lysis buffer (CST, Boston, Mass., USA) and Nuclear and Cytoplasmic Extraction Reagents kit from Thermo Scientific (Asheville, N.C., USA), respectively. For immunoblotting, proteins from whole-cell, cytoplasm, nuclear, and microsomal fractions were separated on SDS-PAGE, then transferred onto a nitrocellulose membrane from GE Healthcare Life Sciences (Buckinghamshire, UK), and incubated with 5% skimmed milk at room temperature for 1 h. Membranes were incubated with the primary antibodies including cPLA₂, p-cPLA₂, COX-1 and p65 (all dilutions 1:1000), and COX-2 (dilution 1:500) from Cell Signaling Technology (Boston, Mass., USA); and mPGES-1 (dilution 1:200) from Cayman Chemical (Ann Arbor, Mich., USA); and β-actin (dilution 1:1000) from Santa Cruz Biotechnology (Santa Cruz, Calif., USA); and TBP (dilution 1:200) was from Abcam (Cambridge, UK) at 4° C. overnight and the band was visualized by incubating the anti-rabbit or anti-mouse secondary antibodies (dilution 1:10,000) from Li-COR (Lincoln, Nebr., USA) at room temperature for 1 h. The levels of protein expression were measured using Odyssey v3.0 software from Li-COR (Lincoln, Nebr., USA).

6. Cytotoxicity Assays, Nitrite Assay and ELISA Assays

RAW264.7 cells and rat peritoneal macrophages were seeded in a 96-well plate at density of 1.4×10⁴ and 1.5×10⁵ cells, respectively. Various concentration of SIN was added to the cells for 1 hour before LPS stimulation. After stimulation with LPS for 18 h (RAW264.7 cells) or 24 h (rat peritoneal macrophages), MTT solution was added to each well and incubated for another 4 hours. 100 μl 10% SDS-HCl solution was added to each well for overnight to dissolve the formazan dye. The optical density was measured at 570 nm against a reference wavelength of 650 nm using a microplate UV/VIS spectrophotometer (Tecan, Mannedorf, Switzerland). The OD value in the normal group (cells were not treated by SIN and LPS) was set as 100%. RAW264.7 cells and rat peritoneal macrophages were seeded in 6-well plates and treated with SIN for 1 h, followed stimulated by LPS for 18 h (RAW264.7 cells) or 24 h (rat peritoneal macrophages). The culture medium was collected and the nitrite concentration was tested by Griess reagent (Promega, USA). The levels of PGE₂, PGI₂, TXA₂ and PGD₂, TNF-α and IL-6 in the culture medium were analysis using ELISA kits according to the manufacturer's instructions.

7. QRT-PCR Assays

Peritoneal macrophages were cultured in 6-well plates and pre-treated with the indicated concentration of SIN for 1 h, followed by LPS stimulation for another 12 h. RNA was isolated from rat peritoneal macrophages using the Nucleospin RNA kit from Macherey-Nagel (Duren, Germany) according to the manufacturer's instructions. RNA concentration derived from each sample was assessed by NanoDrop UV Spectrophotometer from Thermo Scientific (Asheville, N.C., USA). RNA (1 μg) was reverse transcripted using the transcriptor universal cDNA master reagents from Roche Applied Science (Mannheim, Germany). Gene expression quantification was performed by a high-productivity real-time quantitative PCR ViiA™ 7 machine using SYBR Green reagents from Roche Applied Science (Mannheim, Germany) according a standard protocol recommended by the manufacturer and the cycling parameters were as followed: uracil removal incubation (50° C., 2 min), polymerase activation (95° C., 10 min), 40 cycles of denaturation (95° C., 15 s) and annealing/extension (60° C., 30 s) and a melt curve stage (95° C., 15 s, 60° C., 1 min and 95° C., 15 s).

Rat primers used in the research are listed as follows:

β-actin,
(SEQ ID NO: 1)
5′-CGTTGACATCCGTAAAGACC-3′ (sense)
and
(SEQ ID NO: 2)
5′-TAGAGCCACCAATCCACACA-3′ (antisense),
COX-2,
(SEQ ID NO: 3)
5′-CATGATCTACCCTCCCCACG-3′ (sense)
and
(SEQ ID NO: 4)
5′-CAGACCAAAGACTTCCTGCCC-3′ (antisense),
mPGES-1,
(SEQ ID NO: 5)
5′-GCGAACTGGGCCAGAACA-3′ (sense)
and
(SEQ ID NO: 6)
5′-GGCCTACCTGGGCAAAATG-3′ (antisense).

The levels of gene expression were quantitated using the 2(−Delta Delta C(T)) method. The target amount of each mRNA sample was divided by the control gene amount (which was assigned a value of 1 arbitrary unit) to obtain a normalized target value.

8. RNA Interference Experiment of mPGES-1

RAW264.7 cells were transfected with ON-TARGETplus SMARTpool mPGES-1 siRNA (mPGES-1 siRNA) or nonspecific siRNA (NS siRNA) using HiPerFect transfection reagent according to the manufacturer's recommended protocols. After 24 h of transfection, the cells were treated with or without SIN (640 μM) or DEX (0.5 μM) in complete growth medium. After 1 h, all groups were stimulated with LPS (10 ng/mL) for 18 h except the control group. Then the cell culture media were collected and stored at −20° C. for later analysis of the levels of PGE₂. Cells were harvested for qRT-PCR, the mouse primers used are listed as follows:

β-actin,
(SEQ ID NO: 7)
5′-CGGTTCCGATGCCCTGAGGCTCTT-3′ (sense)
and
(SEQ ID NO: 8)
5′-CGTCACACTTCATGATGGAATTGA-3′ (antisense),
mPGES-1,
(SEQ ID NO: 9)
5′-ATGAGGCTGCGGAAGAAGG-3′ (sense)
and
(SEQ ID NO: 10)
5′-GCCGAGGAAGAGGAAAGGATAG-3′ (antisense).

9. Immunofluorescence Assays

RAW 264.7 cells were seeded on glass coverslip at a density 2.0×10⁵ cells/well in 6-well plates and incubated for 24 h. Cells were pretreated with 640 μM SIN for 1 h and followed stimulated by LPS (100 ng/ml) for another 1 h. Rat primary peritoneal macrophages were grown on glass coverslip at a density 4.5×10⁶ cells/well in 6-well plates and pretreated with 640 μM SIN for 1 h, and then challenged with LPS (1 μg/ml) for another 30 min (for NF-κB) or 2 h (for C/EBPβ). Cells were fixed with 4% paraformaldehyde for 30 min at room temperature and then blocked for 1 h with 5% BSA in PBS containing 0.1% Triton X-100. Then incubated with a primary antibodies for overnight and followed by Alexa Fluor 594-labeled goat anti-rabbit IgG. Cells were washed three times, and stained with DAPI for 30 min. After a wash step, cells were fixed on the slide and the images were acquired using a LeicaDM2500 fluorescent microscope (Leica Microsystems GmbH, Wet-zlar, Germany).

10. Electrophoretic Mobility Shift Assay (EMSA) Assays for NF-κB

Nuclear extract proteins (10 μg) for the assay of the DNA binding activity of NF-κB were tested with a biotin-labeled oligonucleotide bio-NF-κB probe according to manufacturer's instructions of EMSA kit (Viagene Biotech).

11. Chromatin Immunoprecipitation Assays (ChIP)

Rat peritoneal macrophages were pretreated with 640 μM SIN (1 h) and stimulated with 1 μg/ml LPS (30 min) before fixation at room temperature for 10 min with 1% formaldehyde. Fixation was terminated by adding glycine (to 0.125 M) with an additional incubation of 5 min, then the cells were washed twice with ice-cold PBS and harvested by scraping and centrifugation (1500 rpm for 10 min at 4° C.). Cell lysis was performed using ChIP kit from abcam (Cambridge, UK) according to the manufacturer's instructions. Briefly, cell pellets were resuspended in 1 ml Buffer B by pipetting up and down several times in a microcentrifuge tube and incubated at room temperature for 10 min. After centrifugation at 5000 rpm, the pellet was collected and resuspended in ice cold Buffer C and incubated on ice for 10 min, followed by centrifugation (5,000 rpm for 10 min at 4° C.). The pellet was mixed with 100 μl Buffer D containing protease inhibitors, incubated on ice for 10 min and sonicated for 30 cycles in a Bioruptor UCD-300 sonicator (power on high, 30 s on, 30 s off per cycle), yielding DNA fragments of 150-200 bps (the size of sonicated chromatin was checked through running 2% agarose gel). Beads were blocked overnight in PBS with 0.5% BSA and then added to the samples. After 2 h incubation at 4° C., beads were removed by centrifugation at 1000 rpm for 5 min at 4° C., the supernatant was transferred and incubated with anti-p65 antibodies (Santa Cruz, Calif., USA)/anti-CEBPβ antibodies (Santa Cruz) at 4° C. overnight, then mixed with 50% washed G slurry and incubated for 4 h, followed by centrifugation (1,000 rpm for 5 min at 4° C.). The supernatant of IgG control was kept as the input. DNA was eluted in elution buffer and crosslinks were reversed by incubation at 65° C. overnight. RNA and protein were digested using RNase A and Proteinase K and DNA was purified by phenol/chloroform/Isoamyl alcohol (25:24:1) extraction and glycogen-ethanol precipitation, and precipitated DNA was resuspended in ddH₂O. Target DNA abundance in ChIP eluate was assayed by qPCR with primer pairs designed to achieve PCR products of 100-200 bps.

Primer sequences used in this study are as follows:

GAPDH,
(SEQ ID NO: 11)
5′-GTGCAAAAGACCCTGAACAATG-3′ (sense)
and
(SEQ ID NO: 12)
5′-GAAGCTATTCTAGTCTGATAACCTCC-3′ (antisense);
NF-κB (mPGES-1 promoter),
(SEQ ID NO: 13)
5′-GAGGGCTGACGAGATAGT-3′ (sense)
and
(SEQ ID NO: 14)
5′-ACTGATGAGGCTGGAGAT-3′ (antisense),
NF-κB (COX-2 promoter),
(SEQ ID NO: 15)
5′-GGAGAGGCAAGGGGATTC-3′ (sense)
and
(SEQ ID NO: 16)
5′-GGAGGAGCAAGAGAATGTCA-3′ (antisense),
CEBPβ (mPGES-1 promoter),
(SEQ ID NO: 17)
5′-GCTCTAGCAAGTTGTTCT-3′ (sense)
and
(SEQ ID NO: 18)
5′-AATTGCCTGGCTTATCTT-3′ (antisense);
CEBPβ (COX-2 promoter),
(SEQ ID NO: 19)
5′-TCTCTTGGCACCACTTTG-3′ (sense)
and
(SEQ ID NO: 20)
5′-ATAGGGGCAGGCTTTACT-3′ (antisense).

12. Statistical Analysis

Data were presented as mean±S.E.M or mean±S.D. ChIP assay results were statistically evaluated using the Independent-Samples T test. Others data statistical differences were calculated with one-way analysis of variance (ANOVA) using SPSS 13.0 statistical software. In all cases, a level of p<0.05 was considered statistically significant.

### Example 1

### Effects of Sinomenine on Inhibiting mPGES-1 Expression in LPS-Activated Macrophages and IL-1β-Stimulated A549 Cells

The inventors examined the effects of the sinomenine (SIN) on PGE₂ production in LPS-stimulated rat peritoneal macrophages and RAW264.7 cells.

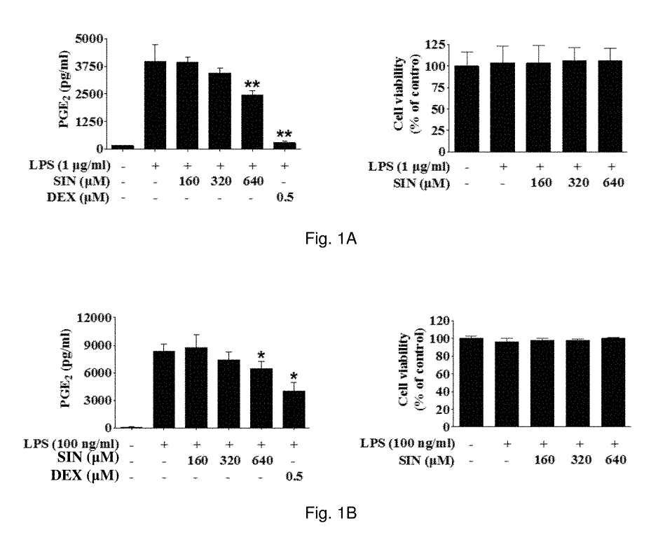

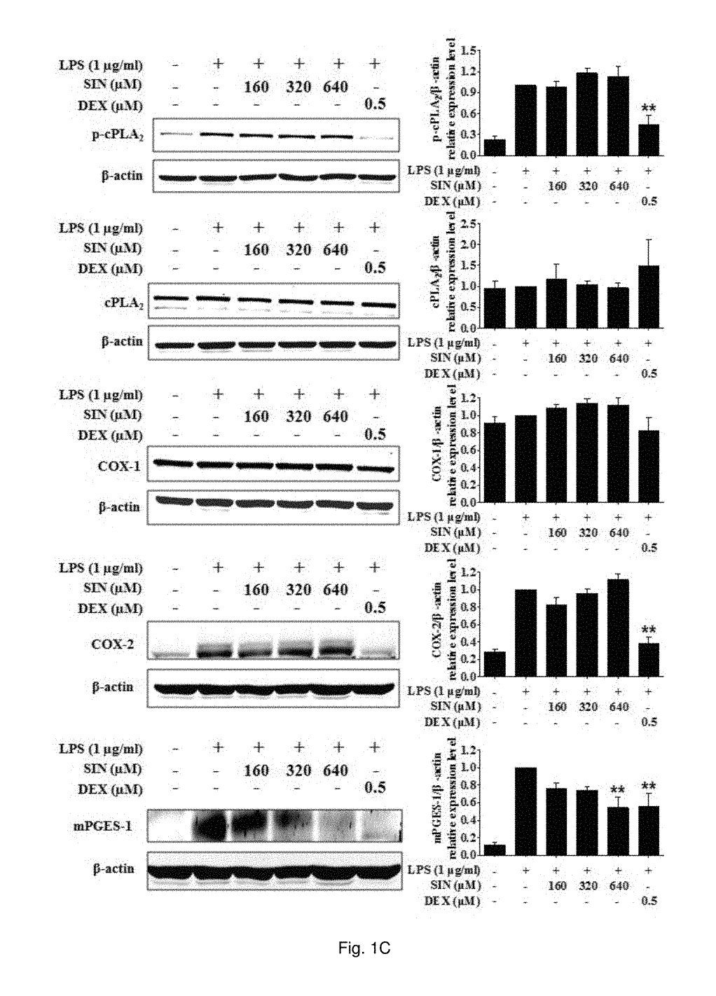

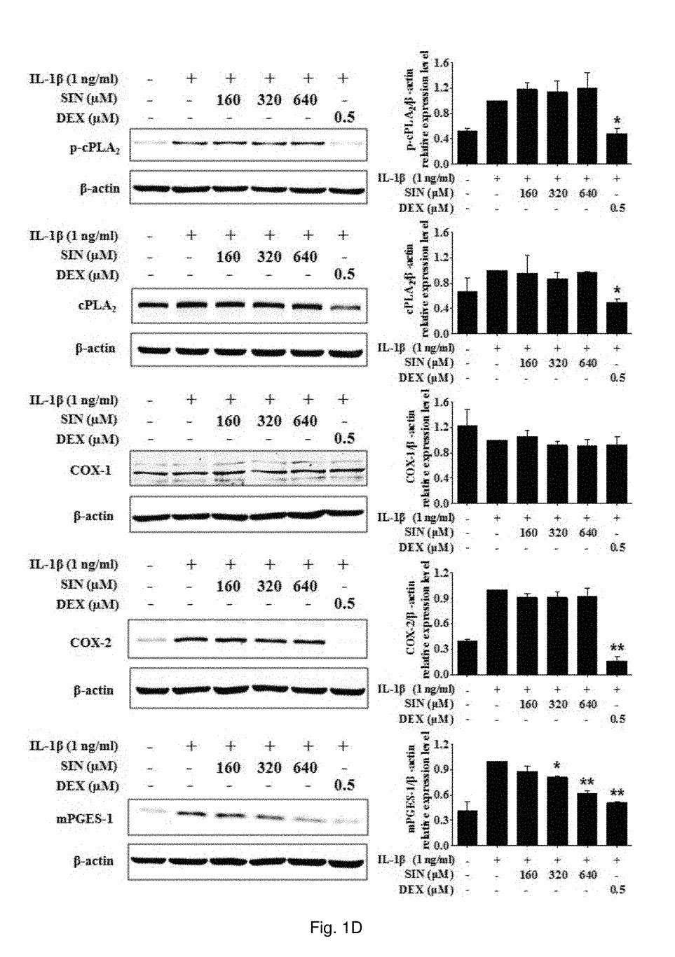

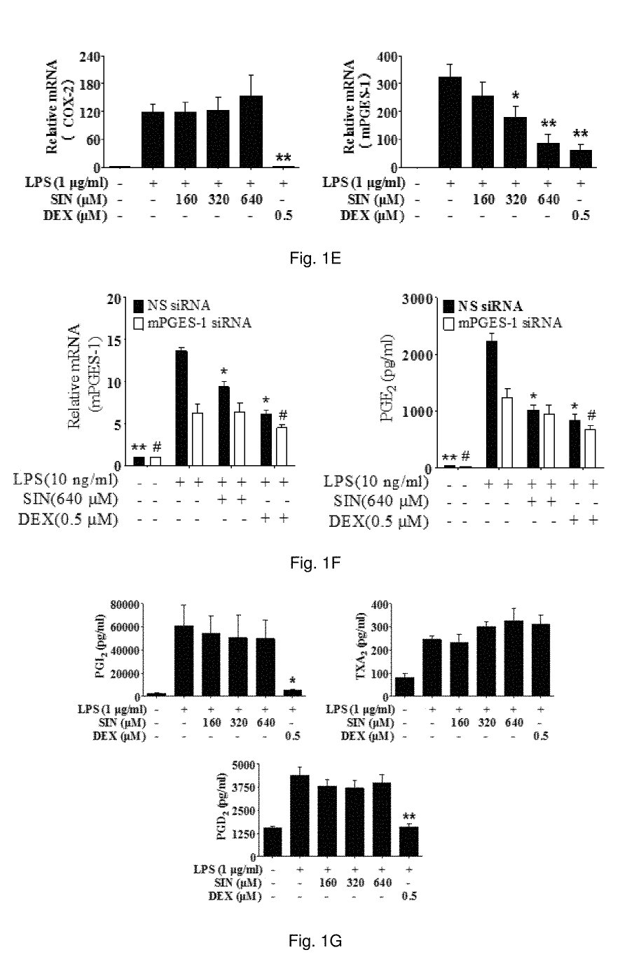

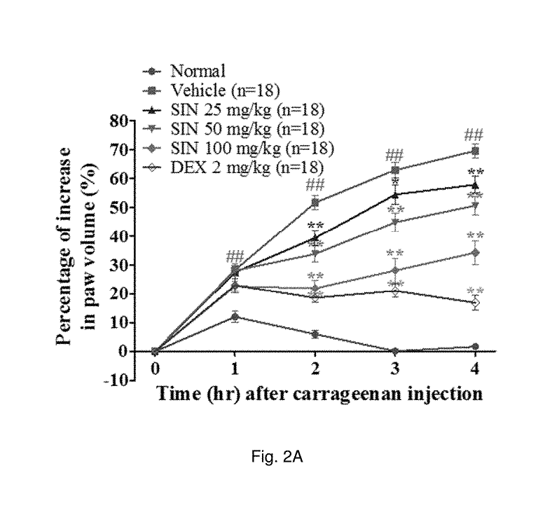

With reference to FIGS. 1A and 1B, the results showed that pretreatment with SIN induced significant inhibition of PGE₂ production in a dose dependent manner. Further studies in LPS-activated rat peritoneal macrophages and IL-1β-stimulated A549 cells showed, in FIGS. 10 and 1D, that SIN did not suppress p-cPLA₂, COX-1 and COX-2 protein expression, as well as the gene level of COX-2, in FIG. 1E, but SIN significantly and dose-dependently inhibited mPGES-1 gene and protein expression. Dexamethasone (DEX), a classic anti-inflammatory drug in clinic, which down-regulates these mediators' expressions was also applied in the experiments. Selective inhibition on mPGES-1 expression decreases PGE₂ production was proved through RNA interference experiment of mPGES-1. In mPGES-1 knockdown RAW264.7 cells, the inventors found that LPS-induced PGE₂ production remarkably decreased while the inhibitory effect of SIN on PGE₂ production was not affected. However, the suppressive effect of DEX on PGE₂ production was significantly enhanced (FIG. 1F). Study on other prostaglandins production in LPS-stimulated rat peritoneal macrophage model demonstrated that the treatment of SIN showed no significant influence on the levels of PGI₂, TXA₂ and PGD₂. However, DEX obviously inhibited the production of both PGI₂ and PGD₂ (refer to FIG. 1G).

Collectively, these in vitro studies suggest that SIN is able to selectively suppress PGE₂ production via down-regulating mPGES-1 expression instead of cyclooxygenases.

### Example 2

### Inhibitory Effects of Sinomenine on mPGES-1 Expression in Animal Models

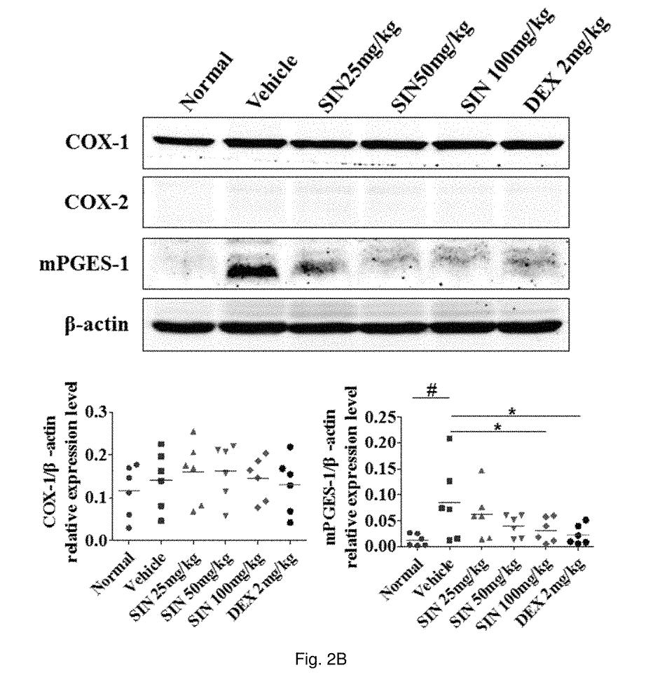

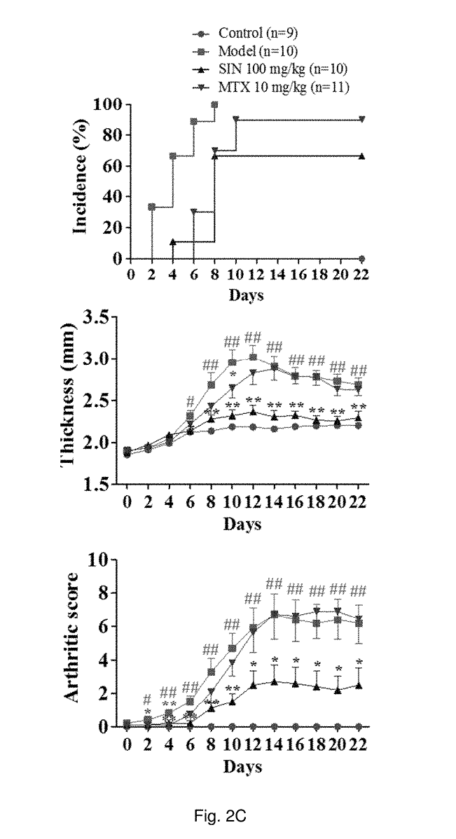

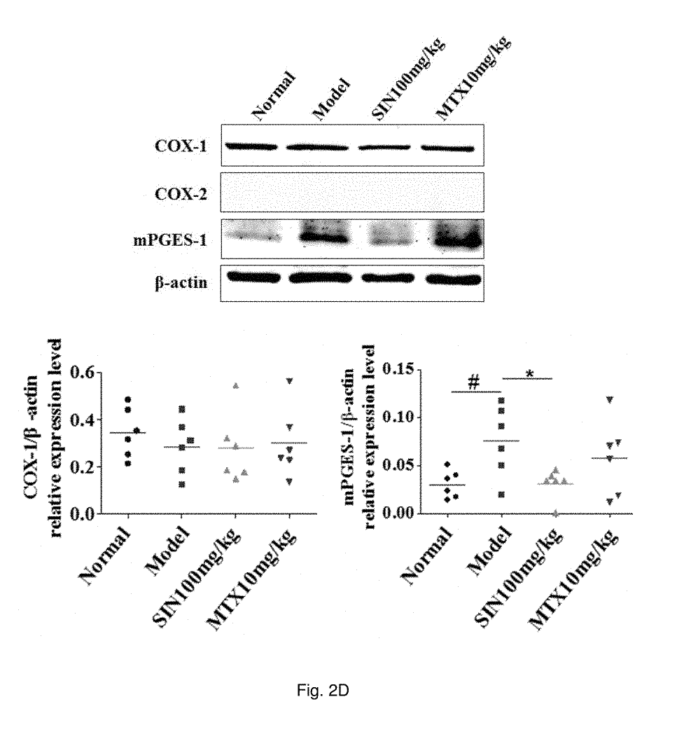

The inventors found that sinomenine is capable of inhibiting mPGES-1 expression in the inflamed paw tissues of mice and rats. First, by using an acute rat inflammatory model, carrageenan-induced rat paw edema, the inventors confirmed the anti-inflammatory potency of SIN (refer to FIG. 2A). In the carrageenan-induced rat paw edema model, swelling of the right hind paws from the vehicle-treated animals occurred rapidly at 1 h after injection of carrageenan in comparison with normal animals, and the swelling continuously increased with time. Pretreatment of SIN with 25, 50 and 100 mg/kg body weight successfully reduced paw edema in a dose dependent manner at 2, 3 and 4 h, however, no effects were seen by SIN at 1 h. Similar to the results of DEX (2 mg/kg), SIN strongly down-regulated the mPGES-1 protein expression in the inflamed paw tissues in a dose dependent manner, but COX-1 protein levels remained unchanged and no COX-2 protein signal was detected in the paw tissues from all animal experimental groups (FIG. 2B).

The inventors further employed a mouse arthritic model, collagen-II induced arthritis (CIA) in DBA/1 mice, to evaluate the anti-arthritic effect of SIN, possibly reflecting the potency of treating RA patients in the clinic. The results showed the incidence of joint swelling in the CIA control group went from 0 on day 0 up to 100% on day 8, while the average thickness of the hind paws of mice not treated with SIN significantly increased from 1.9 mm on day 0 to a maximum of 3.0 mm by day 12. Moreover, the total arthritic scores of four paws also increased, with a maximum value of 6.7 on day 14.

SIN treatment delayed the onset of the joint swelling and decreased average thickness of the inflamed hind paws as well as the arthritic scores (FIG. 2C). At the same time the level of mPGES-1 expression in the inflamed paws influenced by SIN was determined and results showed that SIN significantly decreased mPGES-1 protein expression (FIG. 2D), similar to results observed in the carrageenan-induced rat paw edema model. In CIA model, the inventors used methotrexate (MTX) as the positive control. MTX is an immune system suppressant in treatment of various autoimmune diseases including rheumatoid arthritis, polymyositis and ankylosing spondylitis. MTX showed no observable effects on both the level of mPGES-1 expression and arthritis in CIA mice (FIGS. 2C and 2D).

### Example 3

### Selective Suppression of Sinomenine on NF-κB DNA Binding Activity in mPGES-1 Promoter

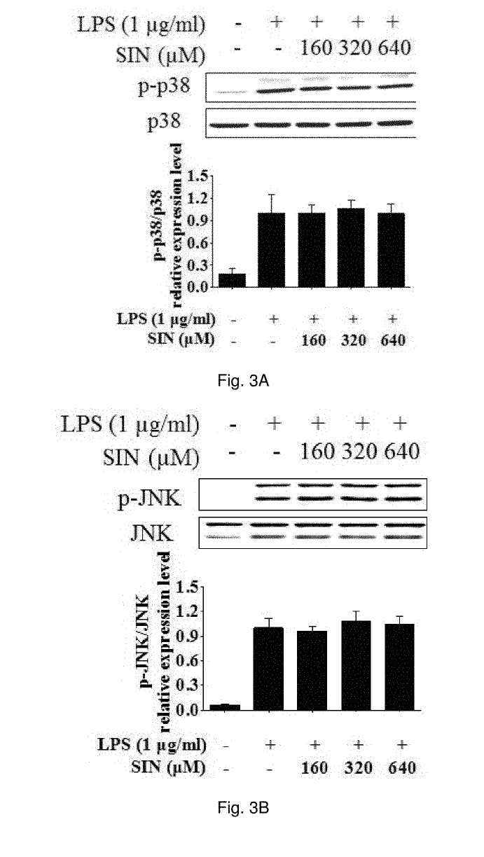

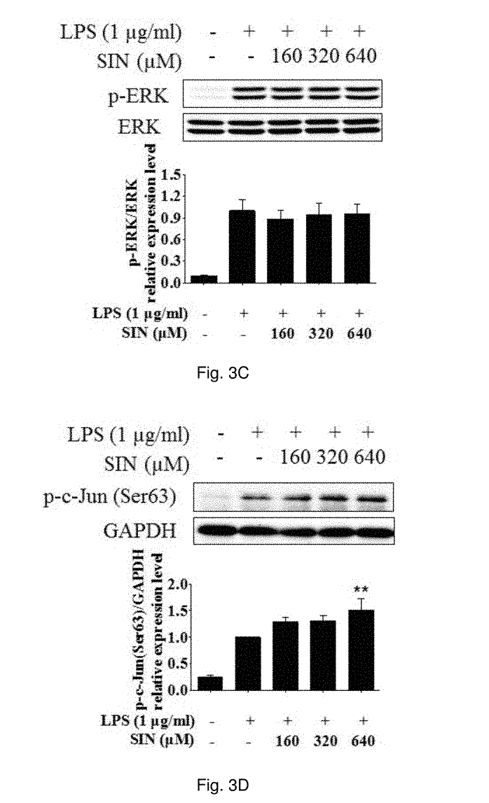

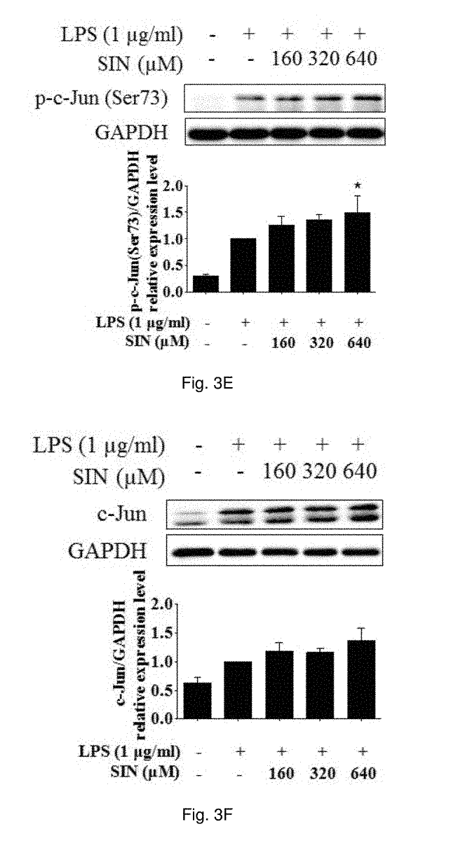

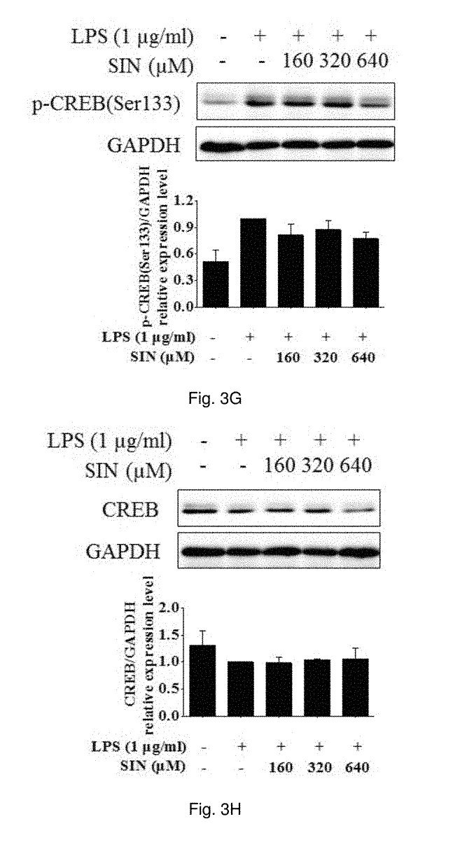

LPS-stimulated rat peritoneal macrophage model was used to investigate the inhibitory effects of sinomenine. Firstly, the inventors investigated the influence of SIN on both mitogen-activated protein kinase (MAPK) and CREB pathways. No observable effects of SIN on these pathways were obtained (refer to FIG. 3A to 3H).

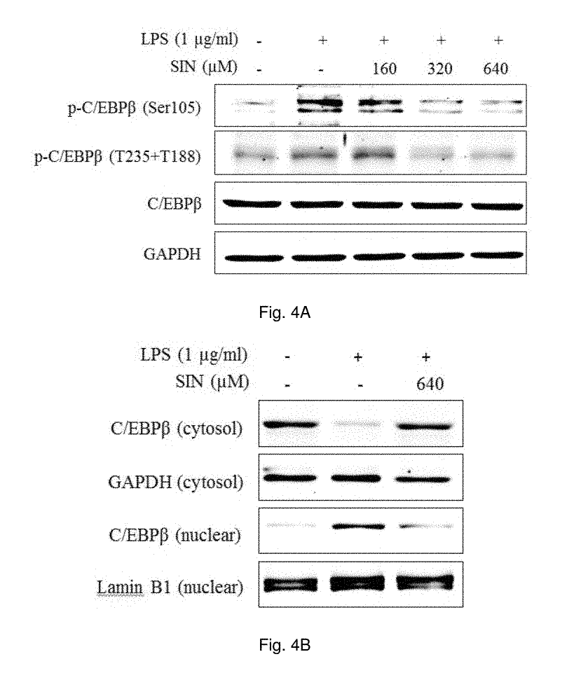

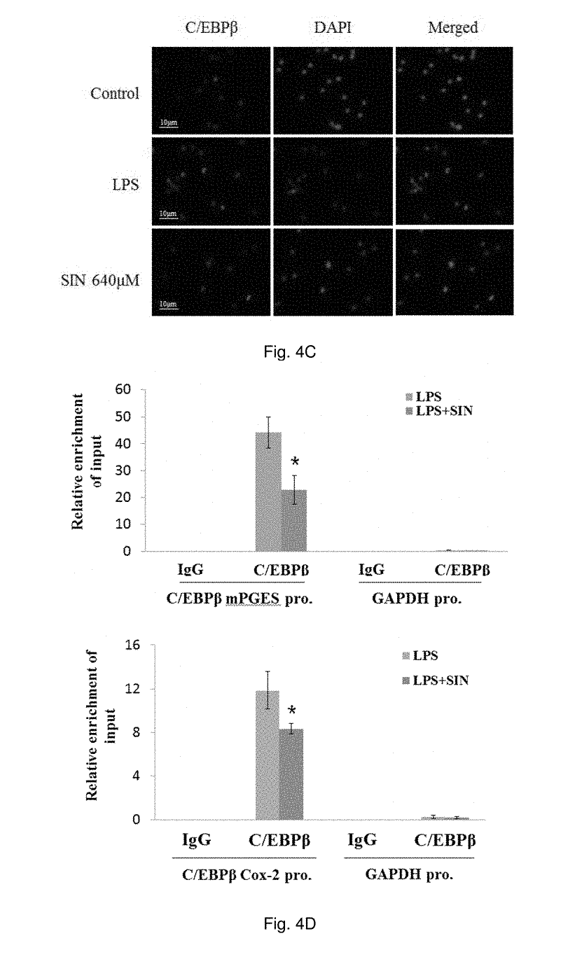

The inventors conducted a study on the effects of SIN on CEBPβ pathway. It was found that SIN exerted effects on C/EBPβ activation (Ser105 and T235+T188) and nuclear translocation of C/EBPβ, as shown in FIG. 4A to 4D. SIN inhibited the DNA binding of CEBPβ to the promoter both of mPGES-1 and COX-2. Accordingly, SIN has inhibitory effects on LPS-induced phosphorylation and nuclear translocation of C/EBPβ, and the C/EBPβ DNA binding activity both in mPGES-1 and COX-2 promoters in macrophages.

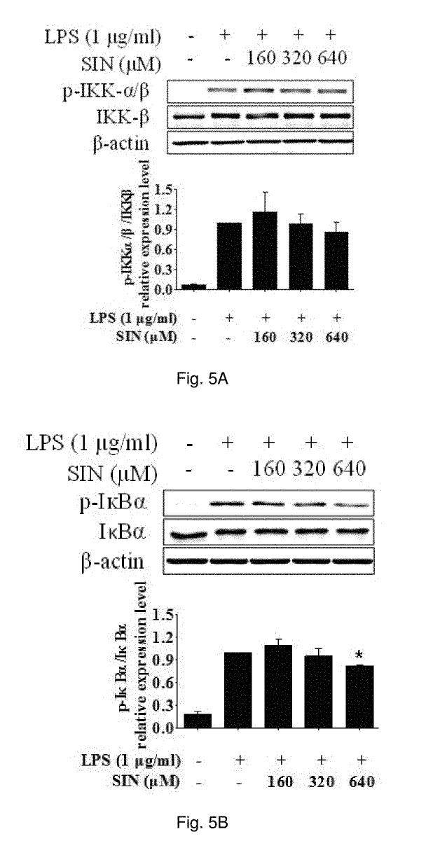

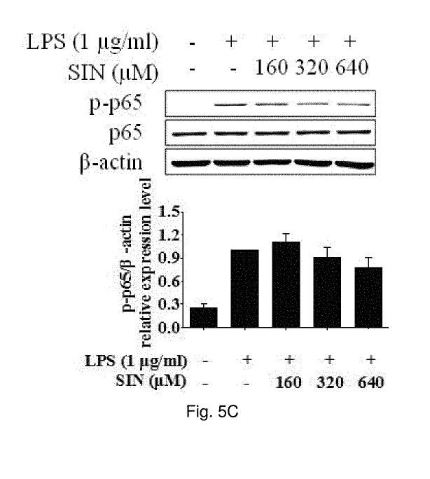

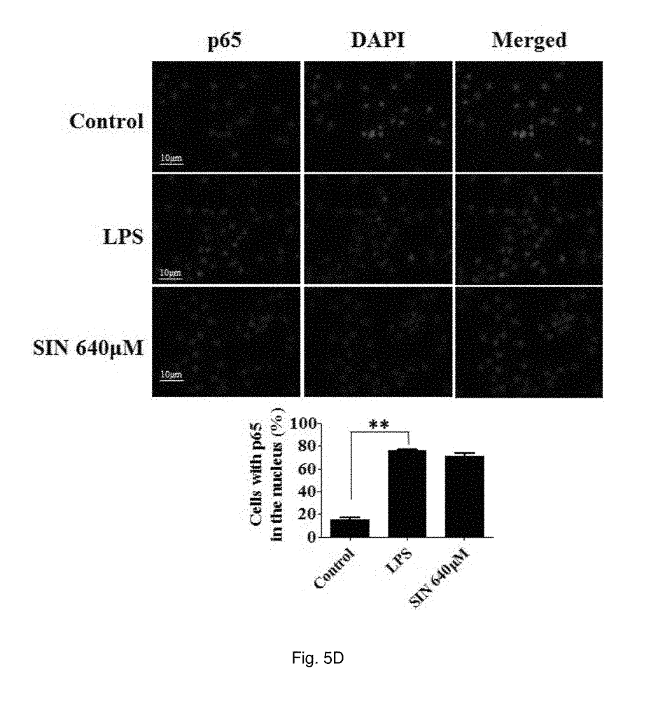

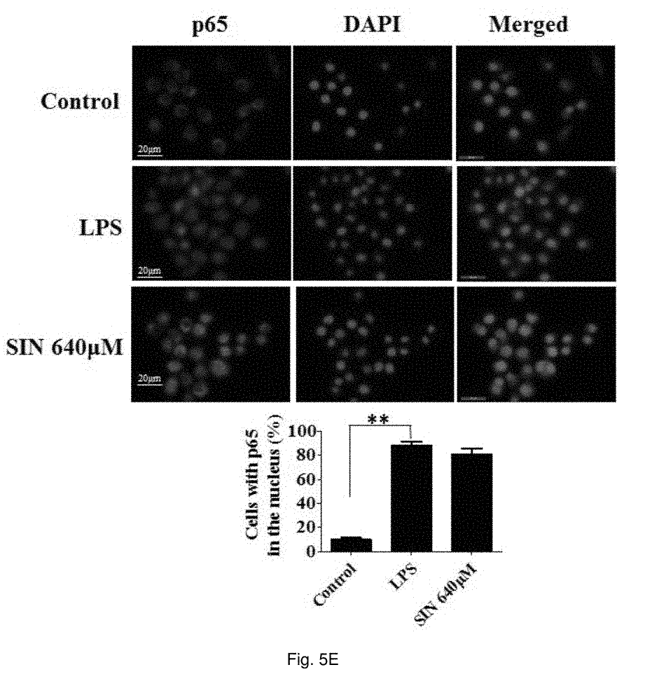

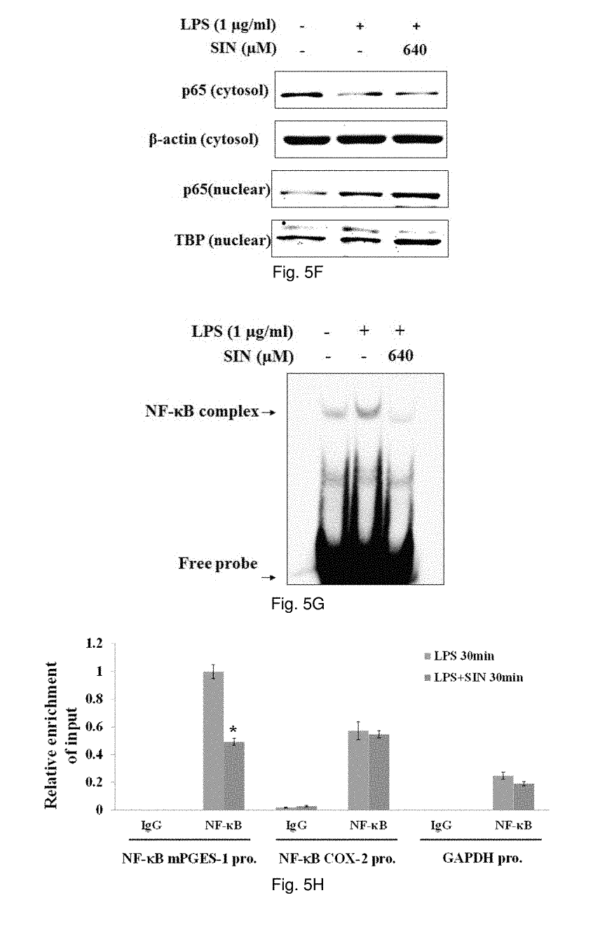

According to FIG. 5A to 5F, SIN does not produce observable inhibitory effects on nuclear translocation of p65 in vitro experiments. However, it is evident from FIG. 5G that SIN is capable of suppressing NF-κB DNA binding activity. ChIP analysis, as shown in FIG. 5H, further demonstrated that SIN selectively inhibited the DNA binding of NF-κB to the promoter of mPGES-1 but not COX-2. Therefore, it demonstrates that SIN can selectively inhibit the mPGES-1 gene expression. Accordingly, SIN is considered an effective compound for preventing and/or treating an inflammatory disease in particular arthritis with lower risk of adverse side effects.

### DISCUSSION

The inventors believe that SIN inhibits PGE₂ release by suppressing mPGES-1 expression, without affecting COX-2 expression.

PGE₂ is the main prostaglandin in the human body and possesses multiple physiological and pathological functions in homeostasis, tissues regeneration, and inflammation. mPGES-1 is the terminal synthases of PGE₂, which catalyzes COX-1 and COX-2-derived PGH₂ conversion to PGE₂ and without affecting the generation of others prostaglandins. Study on in vitro acute inflammatory cells models, DEX was chosen as the positive control, which is a classic anti-inflammatory drug and could immediate inhibition of acute inflammation through the suppression of inflammatory mediators and cytokines production including prostaglandins, nitric oxide (NO), tumor necrosis factor-α (TNF-α), interleukin-6 (IL-6).

In the examples, DEX significantly inhibited NO, TNF-α, IL-6 production as well as iNOS expression induced by LPS in macrophages and decreased PGE₂ release through down-regulating p-cPLA₂, COX-2 and mPGES-1 expression as well as obviously affecting PGI₂ and PGD₂ production. However, SIN is capable of significantly decreasing PGE₂ levels without affecting PGD₂, PGI₂ and TXA₂ synthesis via selectively inhibiting mPGES-1 expression. Without intending to be limited by theory, SIN may reduce cardiovascular risk compared with NSAIDs in particular COX-2 inhibitors currently applied in treatments of inflammatory diseases.

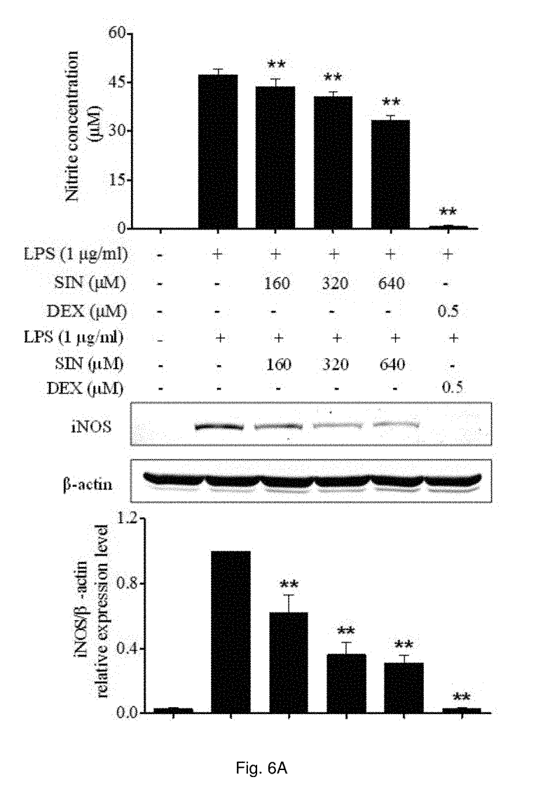

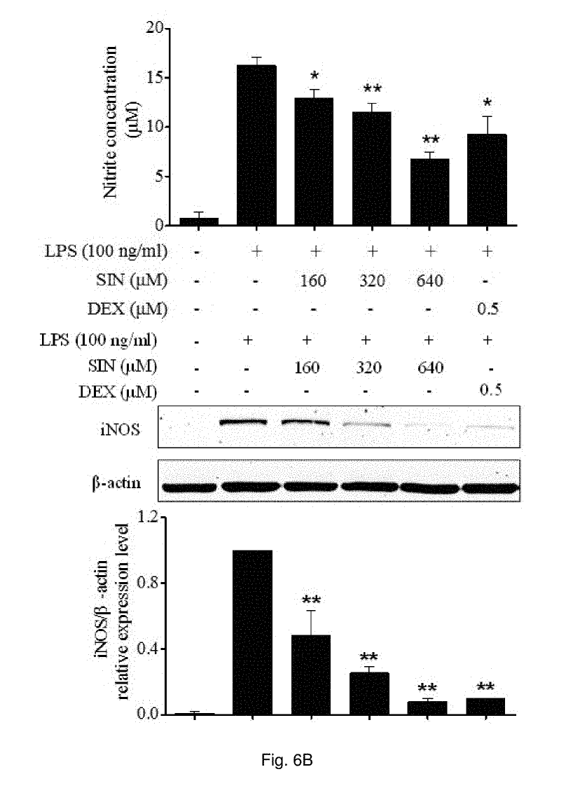

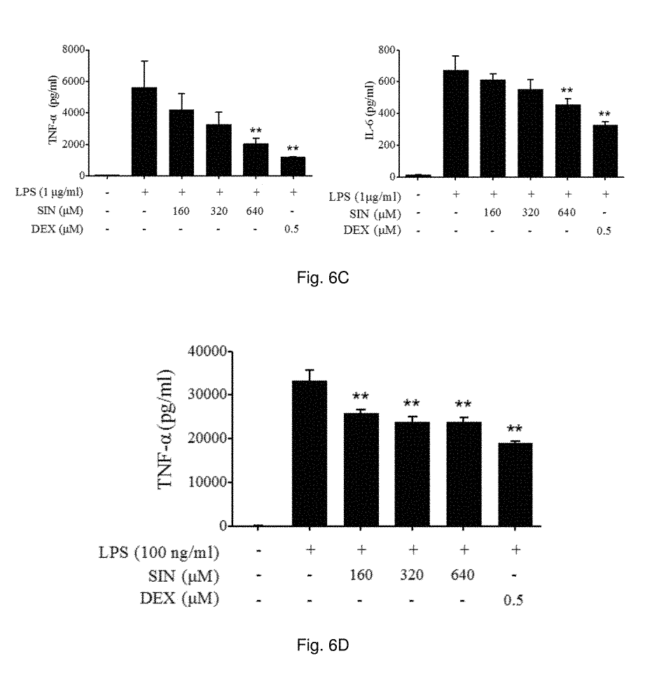

Similar to DEX, SIN also significantly inhibited the release of NO, TNF-α, IL-6 and the expression of iNOS (FIG. 6A to 6D). Results from the RNA interference experiment of mPGES-1 further confirmed that SIN inhibits the release of PGE₂ via the suppression on mPGES-1 expression. In contrast, DEX inhibited the release of PGE₂ through multiple mechanisms. Previous studies reported that DEX may lead to faster heart rates, transient absolute myocardial hypertrophy and increase in systemic blood pressure etc which are considered as cardiac side effects (Evans N. Archives of disease in childhood Fetal and neonatal edition 1994, 70; and Werner J C et al., The Journal of pediatrics 1992, 120). Accordingly, SIN is a potent anti-inflammatory agent and produces fewer side effects as compared to NSAIDs in particular COX-2 inhibitors and steroids in treatment of inflammatory diseases.

It was found that SIN possessed potent anti-inflammatory activity with significant reduction of the paw edema induced by carrageenan and strongly down-regulated the mPGES-1 protein expression in the inflamed paw tissues in a dose dependent manner. In collagen-induced arthritis in DBA/1 mice, the anti-arthritic effect of SIN was also demonstrated with reduction in mPGES-1 protein expression, similar to the results of the carrageenan-induced rat paw edema model.

Both COX-2 and mPGES-1 were often coordinately up-regulated in response to soluble stimuli (such as LPS, tumor necrosis factor-α, interleukin-1β). The inventors found that DEX inhibited both COX-2 and mPGES-1 expressions, but SIN only down-regulated mPGES-1 expression. Nuclear Factor-κB (NF-κB) is a major transcription factor that plays a central role in inflammation by regulating transcription of an array of inflammatory mediators and cytokines. Overexpression of IκBαΔN, an inhibitor protein of NF-κB, repressed IL-1β-induced mPGES-1 expression in A549 cells and transfection of synovial fibroblasts with IκB, reduces the induction of mPGES-1 by microparticles (MPs) in rheumatoid arthritis synovial fibroblasts (RASFs) (Jungel A. et al., Arthritis and rheumatism 2007, 56). These studies indicated that NF-κB play a critical role in inducing mPGES-1 expression. Although both COX-2 and mPGES-1 promoter binding regions contain NF-κB DNA binding sequences, however, the data demonstrated that SIN only selectively inhibits the DNA binding of NF-κB to the mPGES-1 promoter without affecting the DNA binding of NF-κB to the COX-2 promoter, i.e. SIN could selectively inhibit the mPGES-1 expression without affecting COX-2.

Without intending to be limited by theory, it is believed that the selective inhibition of SIN has a reduced risk of cardiovascular side effects compared to drugs currently used such as NSIADs. Furthermore, SIN is capable of selectively decreasing the DNA binding ability of nuclear translocated NF-κB, thus minimizing the interference to the upstream of NF-κB signaling pathway that plays important biological roles in inflammation. The inventors also noted that SIN may be more easily access to the nucleus in vivo experiments than in vitro experiments.

Taken together, the inventors provide a method of selectively inhibiting the overexpression of mPGES-1 in a subject in need thereof in particular a subject suffering from a disease associated with the overexpression of mPGES-1. The selective mPGES-1 inhibitor as disclosed herein is capable of inhibiting the overexpression of mPGES-1 in a mammal and cells, without affecting COX-2 expression.

1. A method of selectively inhibiting the overexpression of mPGES-1 in a subject in need thereof comprising a step of administering an effective amount of a selective mPGES-1 inhibitor having a structure of Formula (I) or a salt thereof to the subject: 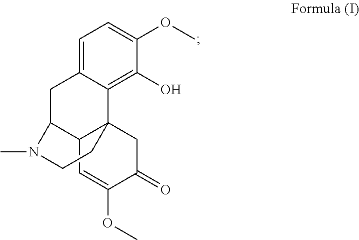
wherein the subject is suffering from a cardiovascular disease or at risk of a cardiovascular event.

2. The method of claim 1, wherein the selective mPGES-1 inhibitor is sinomenine having a structure of Formula (II) or a salt thereof:

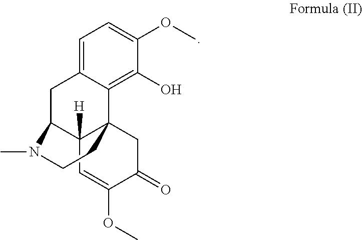

3. The method of claim 1, wherein the subject is suffering from at least one of inflammatory disease, neurological disease, injury, immune disease, gastrointestinal disease, or cancer.

4. The method of claim 3, wherein the subject is suffering from a cardiovascular disease.

5. The method of claim 1, wherein the subject is suffering from arthritis and is at risk of cardiovascular event.

6. The method of claim 5, wherein the cardiovascular event is selected from the group consisting of heart attack, stroke, myocardial infarction, acute coronary syndrome, arteriosclerosis, thrombosis, hypertension, cardiovascular death, and peripheral vascular disease.

7. The method of claim 1, further comprising steps of:
obtaining a sample from the subject;
testing the sample for the expression of mPGES-1;
comparing the level of mPGES-1 expression with a reference to determine if the subject has an overexpression of mPGES-1; and
optionally determining if the subject is suffering from a cardiovascular disease or being at risk of a cardiovascular event.

8. The method of claim 1, wherein the administration of the selective mPGES-1 inhibitor suppresses the binding of nuclear factor κB to mPGES-1 promoter.

9. The method of claim 1, wherein the subject receives or received a long-term treatment of a non-steroidal anti-inflammatory drug.

10. A method of treating a subject suffering from arthritis associated with an overexpression of mPGES-1 and having a risk of cardiovascular event, comprising the step of administering an effective amount of a selective mPGES-1 inhibitor to the subject, wherein the selective mPGES-1 inhibitor has a structure of Formula (II) or a salt thereof:

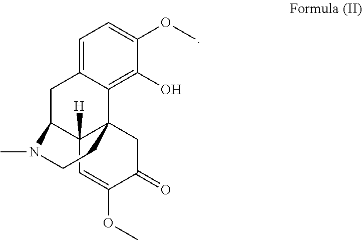

11. (canceled)

12. (canceled)

13. (canceled)
